(19) United States
(12) Patent Application Publication
(10) Pub. No.: US 2020/0123123 A1
(43) Pub. Date: Apr. 23, 2020

(54) PROCESS FOR PREPARING 2,5-FURANDICARBOXYLIC ACID

(71) Applicant: Far Eastern New Century Corporation, Taipei City (TW)

(72) Inventors: Wei-Lin Zheng, Taipei City (TW); Xin-An Lu, Taipei City (TW)

(21) Appl. No.: 16/366,154

(22) Filed: Mar. 27, 2019

(30) Foreign Application Priority Data
Oct. 18, 2018 (TW) 107136674

(51) Int. Cl. C07D 307/68

(52) U.S. Cl. CPC C07D 307/68

(57) ABSTRACT

A process for preparing 2,5-furandicarboxylic acid includes the steps of: subjecting an 5-acyloxymethyl-furfural compound to an oxidation reaction with an oxygen-containing gas in the presence of a basic aqueous solution and an oxidation catalyst containing a metal selected from the group consisting of ruthenium, rhodium, palladium, and combinations thereof to form an aqueous solution containing 2,5-furandicarboxylate, and converting the 2,5-furandicarboxylate to 2,5-furandicarboxylic acid.

### CROSS-REFERENCE TO RELATED APPLICATION

This application claims priority of Taiwanese Application No. 107136674, filed on Oct. 18, 2018.

### FIELD

The disclosure relates to a process for preparing 2,5-furandicarboxylic acid, and more particularly to a process for preparing 2,5-furandicarboxylic acid in the presence of a basic aqueous solution and an oxidation catalyst containing a metal selected from the group consisting of ruthenium, rhodium, palladium, and combinations thereof.

### BACKGROUND

2,5-furandicarboxylic acid (FDCA) has been suggested as an important renewable building block because of its ability to substitute terephthalic acid in the production of polyesters and other polymers containing an aromatic moiety.

PCT Publication No. WO 2015/056270 discloses a process for preparing 2,5-furandicarboxylic acid. The process comprises the steps of: (i) subjecting 5-hydroxymethylfurfural to an acylation reaction with an acylating agent to form 5-acyloxymethylfurfural; (ii) subjecting the 5-acyloxymethylfurfural to an oxidation reaction with an oxidizing agent in the presence of a reaction solvent to form 5-acyloxymethylfurancarboxylic acid, wherein the oxidizing agent is selected from the group consisting of nitric acid, bromine water, sodium hypochlorite, sodium chlorite, sodium bromite, potassium chlorite, potassium bromite, hydrogen peroxide, potassium permanganate, a combination of sodium chlorite and hydrogen peroxide, and a combination of potassium chlorite and hydrogen peroxide, and the reaction solvent is selected from the group consisting of acetonitrile, tetrahydrofuran, ethyl acetate, and chloroform; (iii) subjecting the 5-acyloxymethylfurancarboxylic acid to an oxidation reaction with an oxidizing agent to form 2,5-furandicarboxylic acid, wherein the oxidizing agent used in step (iii) is the same as that used in step (ii), and the oxidation reaction in step (iii) can be implemented in the presence or absence of water. The yield of the 2,5-furandicarboxylic acid obtained by this process is at least 70%. However, the 5-hydroxymethylfurfural has unstable chemical properties, and is difficult to be purified, stored, and transported. In addition, since the preparation process of the 2,5-furandicarboxylic acid involves many steps, it is difficult to avoid a high production cost.

U.S. Pat. No. 8,865,921 discloses a method for preparing 2,5-furandicarboxylic acid in high yield. The method includes the step of contacting a feed comprising a compound selected from the group consisting of 5-hydroxymethylfurfural, an ester of 5-hydroxymethyl-furfural, 5-methylfurfural, 5-(chloromethyl)furfural, 5-methylfuroic acid, 5-(chloromethyl)furoic acid, 2,5-dimethylfuran, and a mixture of two or more of these compounds with an oxidant in the presence of an oxidation catalyst at a temperature higher than 140° C. The oxidation catalyst comprises cobalt (Co), manganese (Mn), and a source of bromine (for example, bromide), and optionally, at least one additional metal (i.e., zirconium (Zr) and/or cerium (Ce)). Solvents suitable for use in the method preferably have at least one component that contains a monocarboxylic acid functional group. A preferred solvent is an aliphatic C₂-C₆ monocarboxylic acid, such as acetic acid, propionic acid, n-butyric acid, isobutyric acid, n-valeric acid, trimethylacetic acid, caproic acid, and mixtures thereof. The mixtures may also include benzene, acetonitrile, heptane, acetic anhydride, chlorobenzene, o-dichlorobenzene, and water. The oxidant used in the method is preferably an oxygen-containing gas or gas mixture, such as air and oxygen-enriched air. The yield of the 2,5-furandicarboxylic acid obtained by this method is in a range from 46% to 64%. However, the monocarboxylic acid is corrosive, and thus may damage the equipment used for preparing the 2,5-furandicarboxylic acid. In addition, the method is implemented at high pressure and high temperature, which may lead to explosion, especially when acetic acid is used as the solvent. In addition, when organic solvent such as benzene is included in the solvent, additional cost is required for the treatment and disposal of a waste resulting from the organic solvent.

U.S. Pat. No. 9,388,152 discloses a process for synthesizing 2,5-furandicarboxylic acid. The process comprises the step of carrying out an oxidation reaction by bringing the following compounds into contact: a) a composition containing furan-2,5-dialdehyde (DFF), b) a protic solvent, c) a source of oxidizing agent, and d) a metallic oxidation catalyst. The oxidative reaction is carried out at a temperature between 40° C. and 60° C. The metallic oxidation catalyst is based on a metal selected from the group consisting of ruthenium (Ru), manganese (Mn), chromium (Cr), molybdenum (Mo), zinc (Zn), iron (Fe), copper (Cu), and vanadium (V). The protic solvent is selected from the group consisting of water, alcohols, organic acids, and mixtures thereof. The oxidizing agent is selected from the group consisting of an aqueous hydrogen peroxide solution, peracetic acid, and a mixture thereof. The yield of the 2,5-furandicarboxylic acid obtained by this process is greater than 70%. However, when the organic acid is used as the protic solvent, it can cause damage to the equipment used for synthesizing the 2,5-furandicarboxylic acid due to the corrosive property of the organic acid. In addition, since the process is implemented at high pressure and high temperature, a risk of explosion exists, especially when the protic solvent is acetic acid. Moreover, when the alcohol is used as the protic solvent, additional cost is required for the treatment and disposal of a waste resulting from the alcohol.

PCT Publication No. WO 2018/017382 discloses an oxidation process to prepare a crude carboxylic acid product. The process comprises oxidizing a feed stream comprising at least one oxidizable compound to generate a crude carboxylic acid slurry comprising furan-2,5-dicarboxylic acid (FDCA) and compositions thereof.

U.S. Pat. No. 9,206,149 discloses a process to produce a carboxylic acid composition. The process comprises a step of oxidizing an oxidizable compound in an oxidizable raw material stream in the presence of a solvent stream, an oxidizing gas stream, and a catalyst system, which is conducted in an primary oxidation zone, to produce a carboxylic acid composition comprising furan-2,5-dicarboxylic acid (FDCA). The oxidizable raw material stream comprises at least one compound selected from the group consisting of 5-(hydroxymethyl)furfural (5-HMF), 5-HMF esters, 5-HMF ethers, 5-alkyl furfurals, mixed feedstocks of 5-HMF and 5-HMF esters, mixed feedstocks of 5-HMF and 5-HMF ethers, and mixed feedstocks of 5-HMF and 5-alkyl furfurals. The catalyst system comprises cobalt, manganese, and bromine. The oxidation is accomplished at a temperature ranging from 100° C. to 220° C. The solvent stream includes water, aliphatic carboxylic acids, and combinations thereof. The oxidizing gas stream includes air and purified oxygen. The yield of 2,5-furandicarboxylic acid obtained by this process is at least 88%. However, when the aliphatic carboxylic acid is used as the solvent stream, it can cause damage to the equipment used for implementing the process due to the corrosive property of the aliphatic carboxylic acid. In addition, since the process is implemented at high pressure and high temperature, a risk of explosion exists, especially when the aliphatic carboxylic acid used in the solvent stream is acetic acid.

U.S. Pat. No. 8,558,018 discloses a process for oxidation of furan aldehydes such as 5-(hydroxymethyl)furfural (HMF) and derivatives thereof such as 5-(aryloxymethyl) furfural compounds in the presence of dissolved oxygen and a Co(II), Mn(II), Ce(III) salt catalyst or mixtures thereof. The yield of 2,5-furandicarboxylic acid obtained by this process is 54%. However, since organic solvent is used in the process, additional cost is required for the treatment and disposal of a waste resulting from the organic solvent.

A research article entitled “From Lignocellulosic Biomass to Furans via 5-Acetoxymethylfurfural as an Alternative to 5-Hydroxymethylfurfural” reported by Kang E. S. et. al. (2015), Chemsuschem, 8: 1179-1188, discloses transformation of 5-acetoxymethylfurfural (AMF) into 2,5-furandicarboxylic acid (FDCA), in which Pt/C (i.e. platinum carried on activated carbon) is added to a solution of AMF in a saturated aqueous NaHCO₃ solution, followed by stirring with oxygen molecules bubbling through the liquid aqueous phase for 2 hours at 70° C. However, this process incurred a high production cost since Pt is used as a catalyst, and a risk of explosion exists due to the use of the oxygen molecules.

### SUMMARY

An object of the disclosure is to provide a process for preparing 2,5-furandicarboxylic acid with reduced production cost.

According to the disclosure, there is provided a process for preparing 2,5-furandicarboxylic acid, comprising the steps of:

(a) subjecting an 5-acyloxymethyl-furfural compound of Formula (I) to an oxidation reaction with an oxygen-containing gas in the presence of a basic aqueous solution and an oxidation catalyst containing a metal selected from the group consisting of ruthenium, rhodium, palladium, and combinations thereof to form an aqueous solution containing 2,5-furandicarboxylate,

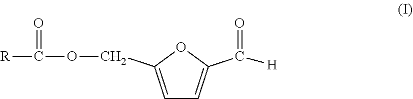

wherein R is selected from the group consisting of hydrogen and a C₁-C₉ hydrocarbyl group; and

(b) converting the 2,5-furandicarboxylate to 2,5-furandicarboxylic acid.

In step (a), the 2,5-furandicarboxylic acid produced from the oxidation reaction in the presence of the basic aqueous solution is neutralized with a basic compound included in the basic aqueous solution to form the 2,5-furandicarboxylate, which can be dissolved in water. Therefore, the process for preparing 2,5-furandicarboxylic acid of this disclosure overcomes the aforesaid shortcomings encountered in the prior art, such as equipment corrosion, explosion risk, and waste treatment and disposal. Specifically, since the oxidation reaction is implemented in the presence of the basic aqueous solution, the process according to the disclosure can be conducted in a general reaction tank made from, for example, 316 stainless steel (a relatively cheap material), rather than in a specified reaction tank made from, for example, titanium alloy or zirconium alloy (a relatively expensive material). Therefore, the cost for the equipment for conducting the process of the disclosure can be reduced.

### DETAILED DESCRIPTION

A process for preparing 2,5-furandicarboxylic acid according to the disclosure comprises a step of:

(a) subjecting an 5-acyloxymethyl-furfural compound of Formula (I) to an oxidation reaction with an oxygen-containing gas in the presence of a basic aqueous solution and an oxidation catalyst containing a metal selected from the group consisting of ruthenium, rhodium, palladium, and combinations thereof to form an aqueous solution containing 2,5-furandicarboxylate,

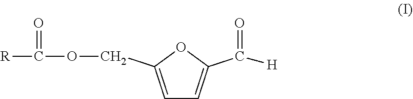

wherein R is selected from the group consisting of hydrogen and a C₁-C₉ hydrocarbyl group; and

(b) converting the 2,5-furandicarboxylate to 2,5-furandicarboxylic acid.

### Oxidation Catalyst:

The oxidation reaction in step (a) is implemented in a gas-liquid-solid reaction system. Specifically, the oxidation catalyst is in a solid form, and further contains a support for carrying the metal. Examples of the support include, but are not limited to, activated carbon, alumina, and a combination thereof.

Although the metal (i.e., ruthenium, rhodium, and/or palladium) contained in the oxidation catalyst in this disclosure has a relatively low activity as compared to platinum used as a catalytic metal in the aforesaid prior art, use of the metal such as ruthenium, rhodium, palladium, or combinations thereof together with specific reaction conditions (such as in the presence of the basic aqueous solution) can effectively enhance the reaction efficiency in the process for preparing 2,5-furandicarboxylic acid and thus the yield thereof. In addition, when ruthenium is used in the oxidation catalyst in the process for preparing 2,5-furandicarboxylic acid, the production cost can be reduced as compared to that of using platinum as the catalytic metal.

### Basic Aqueous Solution:

In order to avoid a side reaction (for example, an aldol condensation) of 5-acyloxymethyl-furfural compound of Formula (I) caused by the basic aqueous solution during the oxidation reaction, and to obtain a relatively high selectivity of 2,5-furandicarboxylic acid, it is necessary for the basic aqueous solution to have a pH value ranging from 7.5 to 11.5.

The basic aqueous solution is prepared by dissolving a basic compound in water. Examples of the basic compound include, but are not limited to, alkali metal carbonates, alkali metal hydrocarbonates, alkaline earth metal carbonates, alkaline earth metal hydrocarbonates, alkali metal hydroxide, and alkaline earth metal hydroxides. Examples of the alkali metal carbonates include, but are not limited to, sodium carbonate and potassium carbonate. Examples of the alkaline earth metal carbonates include, but are not limited to, calcium carbonate and magnesium carbonate. Examples of the alkali metal hydrocarbonates include, but are not limited to, sodium hydrocarbonate and potassium hydrocarbonate. A non-limiting example of the alkali metal hydroxides is sodium hydroxide. Examples of the alkaline earth metal hydroxides include, but are not limited to, calcium hydroxide and magnesium hydroxide. The aforesaid examples of the basic compound can be used alone or as a mixture of two or more.

Since 2,5-furandicarboxylic acid is not easily dissolved in water, a solid form thereof may poison the oxidation catalyst during the oxidation reaction, which may lead to reduction of yield, selectivity, and conversion of 2,5-furandicarboxylic acid. The basic compound used in step (a) is capable of reacting with 2,5-furandicarboxylic acid produced by the oxidation reaction to form 2,5-furandicarboxylate, which can be dissolved in water so as to avoid poisoning the oxidation catalyst. In addition, no organic solvent is used in the process for preparing 2,5-furandicarboxylic acid according to the disclosure, and thus, a further treatment of a waste resulting from the organic solvent is unnecessary.

### 5-Acyloxymethyl-Furfural Compound of Formula (I):

In certain embodiments, a molar ratio of the 5-acyloxymethyl-furfural compound of Formula (I) to the metal of the oxidation catalyst is less than 40 in order to obtain a high selectivity of 2,5-furandicarboxylic acid prepared according to the disclosure. In certain embodiments, the molar ratio of the 5-acyloxymethyl-furfural compound of formula (I) to the metal of the oxidation catalyst is at least 10 and less than 40. Examples of the 5-acyloxymethyl-furfural compound of formula (I), include, but are not limited to, 5-acetoxymethyl-furfural and 5-octanoyloxymethyl-furfural.

### Oxygen-Containing Gas:

Examples of the oxygen-containing gas include, but are not limited to, air and pure oxygen. In certain embodiments, the oxygen-containing gas contains oxygen in an amount of at least 1 vol % and less than 100 vol % based on a total volume of the oxygen-containing gas, so as to reduce the production cost. In certain embodiments, the oxygen-containing gas is air.

### Oxidation Reaction:

In certain embodiments, the oxidation reaction is implemented at a pressure ranging from 10 kg/cm² to 30 kg/cm². In certain embodiments, the oxidation reaction is implemented at a temperature ranging from 100° C. to 160° C.

Examples of the disclosure will be described hereinafter. It is to be understood that these examples are exemplary and explanatory and should not be construed as a limitation to the disclosure.

### Example 1

A basic aqueous solution composed of sodium hydrocarbonate (18 g) and deionized water (600 ml) was added into an autoclave. 5-acetoxymethylfurfural (6.7 g, 0.039 mole, 97% purity) and a wetted powdery oxidation catalyst (8.94 g, Ru/C (i.e., ruthenium carried on activated carbon), ruthenium content: 5 wt % based on 100 wt % of the oxidation catalyst, manufacturer: EVONIK, moisture content: 55.9%) were then added into the autoclave, followed by mixing the oxidation catalyst with the basic aqueous solution. Air was then introduced into the autoclave at a flow rate of 12 L/min, followed by an oxidation reaction at a temperature of 130° C. and a pressure of 20 kg/cm² for 4 hours to form a reaction product. The temperature of the autoclave was reduced to room temperature (about 25° C.) and the pressure in the autoclave was released. The reaction product was then poured from the autoclave, followed by filtration of the reaction product with a filter paper to obtain a filter cake. The filter cake was rinsed with deionized water (50 ml) to obtain a light yellow filtrate containing sodium 2,5-furandicarboxylate (660 g).

An aqueous hydrochloric acid solution (1 N) was added to the light yellow filtrate to convert 2,5-furandicarboxylate to 2,5-furandicarboxylic acid so as to obtain a solution containing 2,5-furandicarboxylic acid.

### Examples 2 to 12

The procedure of each of Examples 2 to 12 was similar to that of Example 1, except that the reactants and reaction conditions shown in Tables 1 and 2 were used. The wetted, powdery oxidation catalysts used in Examples 2 to 4 were in amounts of 8.25 g, 6.62 g, and 4.47 g, respectively. The oxidation catalyst used in each of Examples 5 and 6 was a dry powdery oxidation catalyst (Manufacturer: BASF, Model: Ru/Ap ESCAT 44, Ru/Al₂O₃ (i.e., ruthenium carried on alumina), ruthenium content: 5 wt % based on 100 wt % of the oxidation catalyst). The oxidation catalyst used in each of Examples 7 and 8 was a dry powdery oxidation catalyst (Manufacturer: ACROS, Pd/C (i.e., palladium carried on activated carbon), palladium content: 10 wt % based on 100 wt % of the oxidation catalyst). The oxidation catalyst used in Example 12 was a dry powdery oxidation catalyst (Manufacturer: Alfa Aesor, Rh/C (i.e., rhodium carried on activated carbon), rhodium content: 5 wt % based on 100 wt % of the oxidation catalyst).

### Comparative Example 1

The procedure of Comparative Example 1 was similar to that of Example 1 with the following exceptions. Water (600 g) was used in place of the basic aqueous solution. The filter cake with white crystal formed thereon was rinsed with methanol (200 ml) to dissolve the white crystal so as to obtain a filtrate (830 g) containing 2,5-furandicarboxylic acid, rather than 2,5-furandicarboxylate. Therefore, the conversion of 2,5-furandicarboxylate to 2,5-furandicarboxylic acid using the aqueous hydrochloric acid solution in Example 1 was omitted.

### Comparative Example 2

The procedure of Comparative Example 2 was similar to that of Comparative Example 1, except that an aqueous sulfuric acid solution (0.2 g, 97 wt %) was used in place of the basic aqueous solution.

### Property Evaluation:

### 1. Selectivity of 2,5-Furandicarboxylic Acid (FDCA) (%):

The solution containing FDCA obtained in each of Examples 1 to 12 and the filtrate containing FDCA obtained in each of Comparative Examples 1 and 2 were analyzed using high performance liquid chromatography (HPLC) (a C18 column (Manufacturer: Waters, Model: 186002560); a mixture of an aqueous phosphoric acid solution (0.5 wt %) and acetonitrile in a volume ratio of 4:1 as a mobile phase; and a diode array detector (Manufacturer: Hitachi, Model: L-2455)) to obtain an amount of FDCA (W, in a unit of gram (g)) in each of the solution and the filtrate. Selectivity of the thus obtained FDCA was calculated as follows.

Selectivity of FOCA (%)=(A/B)×100%

wherein

A: yield of FDCA; and

B: conversion of the 5-acyloxymethyl-furfural compound of Formula (I).

The yield of FDCA was calculated as follows.

Yield of FDCA (A)=(W/C)×100%

wherein

W: a measured amount of FDCA from HPLC; and

C: a theoretical yield of FDCA.

The theoretical yield (g) of FDCA was calculated as follows.

Theoretical yield (C)=Y×156.09

wherein

Y: a molar amount of 5-acyloxymethyl-furfural compound of Formula (I); and

156.09: molecular weight of FDCA.

For Examples 1 and 3 to 11 and Comparative Examples 1 and 2, in which 5-acetoxymethylfurfural was used, Y is 0.039 mole (i.e., (6.7×97%)/168.05). For Example 12, in which 5-acetoxymethylfurfural was used, Y is 0.030 mole (i.e., (5.2×97%)/168.05). For Example 2, in which 5-octanoyloxymethyl-furfural was used, Y is 0.036 mole (i.e., (19.98×45%)/252).

### 2. Conversion of the 5-Acyloxymethyl-Furfural Compound of Formula (I) (%):

Conversion of the 5-acyloxymethyl-furfural compound of Formula (I) in each of Examples 1 to 12 and Comparative Examples 1 and 2 was calculated as follows.

Conversion (%)=[(Wₐ−W_{b})/Wₐ]×100%

wherein

Wₐ: an initial amount of 5-acyloxymethyl-furfural compound; and

W_{b}: a remaining amount of 5-acyloxymethyl-furfural compound after the oxidation reaction.

The remaining amount of the 5-acyloxymethyl-furfural compound was measured using the aforesaid HPLC.

### 3. Selectivity of 5-Formal-2-Furancarboxylic Acid (FFCA)(%):

The solution containing FDCA obtained in each of Examples 1 to 12 and the filtrate containing FDCA obtained in each of Comparative Examples 1 and 2 were analyzed using the aforesaid HPLC to obtain an amount of FFCA (W₁, in a unit of gram (g)) in each of the solution and the filtrate. Selectivity of FFCA was calculated as follows.

Selectivity of FFCA (%)=(A₁/B)×100%

wherein

A₁: yield of FFCA; and

B: conversion of the 5-acyloxymethyl-furfural compound of Formula (I).

The yield of FFCA was calculated as follows.

Yield of FFCA (A₁)=(W₁/M_{F})/C₁)×100%

wherein

W₁: a measured amount of FFCA from HPLC;

M_{F}: a molecular weight of FFCA; and

C₁: a theoretical molar amount of FFCA.

Theoretical yield (g) of FFCA was calculated as follows.

Theoretical yield=Y×140.09

wherein

Y: a molar amount of 5-acyloxymethyl-furfural compound of Formula (I); and

140.09: molecular weight of FFCA.

For Examples 1 and 3 to 11 and Comparative Examples 1 and 2, in which 5-acetoxymethylfurfural was used, Y is 0.039 mole (i.e., (6.7×97%)/168.05). For Example 12, in which 5-acetoxymethylfurfural was used, Y is 0.030 mole (i.e., (5.2×97%)/168.05). For Example 2, in which 5-octanoyloxymethyl-furfural was used, Y is 0.036 mole (i.e., (19.98×45%)/252).

### 4. Selectivity of 5-Hydroxymethyl-2-Furoic Acid (HMFA) (%):

The solution containing FDCA obtained in each of Examples 1 to 12 and the filtrate containing FOCA obtained in each of Comparative Examples 1 and 2 were analyzed using the aforesaid HPLC to obtain the amount of HMFA (W₂, in a unit of gram (g)) in each of the solution and the filtrate. Selectivity of HMFA was calculated as follows.

Selectivity of HMFA (%)=(A₂/B)×100%

wherein

A₂: yield of HMFA; and

B: conversion of the 5-acyloxymethyl-furfural compound of Formula (I).

The yield of HMFA was calculated as follows.

Yield of HMFA (A₂)=(W₂/M_{H})/C₂)×100%

wherein

W₂: a measured amount of HMFA from HPLC;

M_{B}: a molecular weight of HMFA; and

C₂: a theoretical molar amount of HMFA.

Theoretical yield (g) of HMFA was calculated as follows.

Theoretical yield=Y×142.11

wherein

Y: a molar amount of 5-acyloxymethyl-furfural compound of Formula (I); and

142.11: molecular weight of HMFA.

For Examples 1 and 3 to 11 and Comparative Examples 1 and 2, in which 5-acetoxymethylfurfural was used, Y is 0.039 mole (i.e., (6.7×97%)/168.05). For Example 12, in which 5-acetoxymethylfurfural was used, Y is 0.030 mole (i.e., (5.2×97%)/168.05). For Example 2, in which 5-octanoyloxymethyl-furfural was used, Y is 0.036 mole (i.e., (19.98×45%)/252).

### 5. Selectivity of Other Components (%):

Selectivity of other compounds was calculated as follows.

Selectivity of other compounds=100%−(S₁+S₂+S³)

wherein

S₁: selectivity of FDCA;

S₂: selectivity of FFCA; and

S₃: selectivity of HMFA.

TABLE 1

| | | | Examples | | | | | |
|---|---|---|---|---|---|---|---|---|
| | | | 1 | 2 | 3 | 4 | 5 | 6 |
| 5-acyloxy | R in Formula (I) | | methyl | heptyl | methyl | methyl | methyl | methyl |
| methyl- | Purity (%) | | 97 | 45 | 97 | 97 | 97 | 97 |
| furfural | g | | 6.7 | 19.98 | 6.7 | 6.7 | 6.7 | 6.7 |
| compound of | Mole (×10⁻²) | | 3.9 | 3.6 | 3.9 | 3.9 | 3.9 | 3.9 |
| Formula (I) | | | | | | | | |
| Basic | Water (ml) | | 600 | 600 | 600 | 600 | 600 | 600 |
| aqueous | Basic | NaHCO₃ | 18 | 18 | 18 | 18 | 18 | 18 |
| solution | compound | | | | | | | |
| | (g) | | | | | | | |

| | | | | | | | |
|---|---|---|---|---|---|---|---|
| | pH (±0.3) | 8.0 | 8.0 | 8.0 | 8.0 | 8.0 | 8.0 |
| Oxidation | g | 3.94 | 3.63 | 2.92 | 1.97 | 3.94 | 7.88 |

| | | | |
|---|---|---|---|
| catalyst | Support | Activated carbon | Alumina |

| | | |
|---|---|---|
| Metal | Type | Ruthenium |

| | | | | | | |
|---|---|---|---|---|---|---|
| Mole (×10⁻³) | 1.95 | 1.79 | 1.44 | 0.98 | 1.95 | 3.90 |

| Molar ratio of | | 20 | | 27 | 40 | 20 | 10 |
|---|---|---|---|---|---|---|---|
| 5-acyloxymethyl-furfural compound | | | | | | | |
| of Formula (I) to metal of oxidation | | | | | | | |
| catalyst | | | | | | | |
| Oxygen- | Type | Air | | | | | |
| containing | Flow rate (L/min) | 12 | | | | | |
| gas | | | | | | | |
| Oxidation | Pressure (kg/cm²) | 20 | | | | | |
| reaction | Temperature (° C.) | 130 | 130 | 130 | 130 | 130 | 130 |
| | Time (hr) | 4 | 4 | 4 | 4 | 4 | 4 |
| Conversion of | | 100 | 100 | 100 | 100 | 100 | 100 |
| 5-acyloxymethyl-furfural compound | | | | | | | |
| of Formula (I) | | | | | | | |
| Selectivity | FDCA | 91.8 | 84.47 | 86.6 | 57.5 | 60.73 | 83.47 |
| | FFCA | — | 1.19 | 0.2 | 14.2 | 0.26 | 0.14 |
| | HMFA | — | — | — | 2.5 | — | — |
| | Others | 8.20 | 14.34 | 13.2 | 25.8 | 39.01 | 16.39 |

—: not measured

TABLE 2

| | | | | | | | | | Comparative | |
|---|---|---|---|---|---|---|---|---|---|---|
| | | | Examples | | | | | | Examples | |
| | | | 7 | 8 | 9 | 10 | 11 | 12 | 1 | 2 |
| 5-acyloxy | R in Formula (I) | | Methyl | | | | | | methyl | |
| methyl- | Purity (%) | | 97 | | | | | | 97 | |
| furfural | g | | 6.7 | | | | | 5.2 | 6.7 | |
| compound of | Mole (×10⁻²) | | 3.9 | | | | | 3.0 | 3.9 | |
| Formula (I) | | | | | | | | | | |
| Basic | Water (ml) | | 600 | | | | | | 600 | |
| aqueous | Basic | NaHCO₃ | 18 | 18 | 0 | 0 | 0 | 18 | 0 | 0 |
| solution | compound | NaOH | 0 | 0 | 0 | 6 | 8.57 | 0 | 0 | 0 |
| | (g) | Na₂CO₃ | 0 | 0 | 11.36 | 7.57 | 0 | 0 | 0 | 0 |

| | | | | | | | | | |
|---|---|---|---|---|---|---|---|---|---|
| | pH (±0.3) | 8.0 | 8.0 | 11.4 | 10.2 | 12.7 | 8.0 | 7.0 | 0 |
| Aqueous | Amount (g) | 0 | 0 | 0 | 0 | 0 | 0 | 0 | 0.2 |
| sulfuric | pH (±0.3) | — | — | — | — | — | — | — | 2.4 |
| acid | | | | | | | | | |
| solution | | | | | | | | | |
| (97 wt %) | | | | | | | | | |

| | | | | | | |
|---|---|---|---|---|---|---|
| Oxidation | g | 2.07 | 4.15 | 3.94 | 3.09 | 3.94 |

| catalyst | Support | | Activated carbon | | | |
|---|---|---|---|---|---|---|
| | Metal | Type | Pd | Ru | Rh | Ru |

| | | | | | |
|---|---|---|---|---|---|
| Mole (×10⁻³) | 1.95 | 3.90 | 1.95 | 1.50 | 1.95 |

| Molar ratio of | | 20 | 10 | 20 | | | | 20 | |
|---|---|---|---|---|---|---|---|---|---|
| 5-acyloxymethyl-furfural | | | | | | | | | |
| compound of Formula (I) to metal | | | | | | | | | |
| of oxidation catalyst | | | | | | | | | |
| Oxygen- | Type | Air | | | | | | | |
| containing | Flow rate (L/min) | 12 | | | | | | | |
| gas | | | | | | | | | |
| Oxidation | Pressure(kg/cm²) | 20 | | | | | | | |
| reaction | Temperature(° C.) | 130 | 140 | 130 | 130 | 130 | 130 | 130 | 130 |
| | Time(hr) | 4 | 4 | 4 | 4 | 4 | 4 | 4 | 4 |
| Conversion of | | 100 | 100 | 100 | 100 | 100 | 100 | 68.3 | 100 |
| 5-acyloxymethyl-furfural | | | | | | | | | |
| compound of Formula (I) | | | | | | | | | |
| Selectivity | FDCA | 38.5 | 71.1 | 79.3 | 85.4 | 51.0 | 6.33 | 14.4 | 51.63 |
| | FFCA | 0.5 | 0.2 | 0.8 | 0.1 | 0.1 | 9.66 | 40.7 | 33.10 |
| | HMFA | 1.1 | 0.3 | — | — | — | 7.08 | 6.2 | — |
| | Others | 59.9 | 28.2 | 19.9 | 14.5 | 48.9 | 76.93 | 38.7 | 15.27 |

—: not measured

As shown in Tables 1 and 2, in the process for preparing 2,5-furandicarboxylic acid according to the disclosure, 2,5-furandicarboxylate can be obtained effectively via the oxidation reaction in the presence of the basic aqueous solution having a pH value ranging from 7.5 to 12.7 and the oxidation catalyst containing ruthenium, rhodium, or palladium. Specifically, when the oxidation catalyst contains ruthenium, the selectivity of the thus obtained 2,5-furandicarboxylic acid is satisfactory. In addition, in the aforesaid research article entitled “From Lignocellulosic Biomass to Furans via 5-Acetoxymethylfurfural as an Alternative to 5-Hydroxymethylfurfural” reported by Kang E. S. et. al. (2015), Chemsuschem, 8: 1179-1188, the selectivity of 2,5-furandicarboxylic acid obtained using platinum in an amount of 0.1 mole based on 1 mole of 2-acetoxymethylfurfural is 82%. As shown in Example 1, the selectivity of 2,5-furandicarboxylic acid can be as high as 91% when ruthenium is used in an amount of only 0.05 mole based on 1 mole of 2-acetoxymethylfurfural. Therefore, the process for preparing 2,5-furandicarboxylic acid according to the disclosure has a relatively low production cost.

In Comparative Examples 1 and 2, the oxidation reactions were implemented in the presence of water (i.e., a neutral environment) and an aqueous sulfuric acid solution (i.e., an acidic environment), respectively, thereby forming 2,5-furandicarboxylic acid directly. As described above, 2,5-furandicarboxylic acid is not easily dissolved in water and a solid form thereof may poison the oxidation catalyst during the oxidation reaction, which may lead to reduction of yield and selectivity, such that the selectivity of 2,5-furandicarboxylic acid is relatively low and the selectivity of 5-formal-2-furancarboxylic acid is undesirably high. As shown in Examples 1 to 11, the basic compound used in the process for preparing 2,5-furandicarboxylic acid according to the disclosure is capable of reacting with 2,5-furandicarboxylic acid produced by the oxidation reaction to form 2,5-furandicarboxylate, which can be dissolved in water so as to avoid poisoning the oxidation catalyst and thus, the selectivity of 2,5-furandicarboxylic acid is relatively high and the selectivity of 5-formal-2-furancarboxylic acid is relatively low.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

1. A process for preparing 2,5-furandicarboxylic acid, comprising the steps of:
(a) subjecting an 5-acyloxymethyl-furfural compound of Formula (I) to an oxidation reaction with an oxygen-containing gas in the presence of a basic aqueous solution and an oxidation catalyst containing ruthenium to form an aqueous solution containing 2,5-furandicarboxylate, 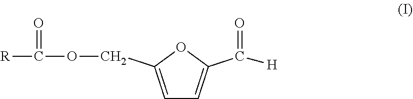
wherein R is selected from the group consisting of hydrogen and a C₁-C₉ hydrocarbyl group; and
(b) converting the 2,5-furandicarboxylate to 2,5-furandicarboxylic acid.

2. The process according to claim 1, wherein the basic aqueous solution has a pH value ranging from 7.5 to 11.5.

3. The process according to claim 1, wherein the oxidation catalyst is in a solid form and further contains a support for carrying ruthenium.

4. The process according to claim 1, wherein a molar ratio of the 5-acyloxymethyl-furfural compound to ruthenium of the oxidation catalyst is less than 40.

5. The process according to claim 3, wherein the support is selected from the group consisting of activated carbon, alumina, and a combination thereof.

6. The process according to claim 1, wherein the oxygen-containing gas contains oxygen in an amount of at least 1 vol % and less than 100 vol % based on a total volume of the oxygen-containing gas.

7. The process according to claim 6, wherein the oxygen-containing gas is air.

8. The process according to claim 1, wherein the basic aqueous solution includes a basic compound selected from the group consisting of sodium hydrocarbonate, sodium carbonate, potassium hydrocarbonate, potassium carbonate, calcium carbonate, magnesium carbonate, calcium hydroxide, magnesium hydroxide, sodium hydroxide, and combinations thereof.

9. The process according to claim 1, wherein the oxidation reaction is implemented at a pressure ranging from 10 kg/cm² to 30 kg/cm².

10. The process according to claim 1, wherein the oxidation reaction is implemented at a temperature ranging from 100° C. to 160° C.
